(19) United States
(12) Patent Application Publication
(10) Pub. No.: US 2015/0031057 A1
(43) Pub. Date: Jan. 29, 2015

(54) METHOD FOR THE DETERMINATION OF RENAL FUNCTION

(71) Applicant: NEUROTUNE AG, Schlieren (CH)

(72) Inventors: Stefan Hettwer, Rupperswil (CH); Jan Willem Vrijbloed, Mohlin (CH); Dominik Stuebl, Munchen (DE); Marcel Roos, Munchen (DE)

(21) Appl. No.: 14/379,887

(22) PCT Filed: Feb. 22, 2013
(86) PCT No.: PCT/EP2013/000521

§ 371 Date: Aug. 20, 2014

(30) Foreign Application Priority Data
Feb. 25, 2012 (EP) 12001255.4

(51) Int. Cl. G01N 33/68

(52) U.S. Cl. CPC G01N 33/6893; G01N 2333/4722; G01N 2333/96433; G01N 2800/347
USPC 435/7.94; 435/23

(57) ABSTRACT

Method for the determination of the renal function wherein the amount of at least one agrin fragment derived by neurotrypsin cleavage of agrin is measured in a sample taken from a patient and the measured amount of the agrin-fragment in the sample is used as indicator for renal function.

The invention refers to a method for determination of the renal function using a special marker.

Serum creatinine and urea are the most widely used biomarkers to monitor kidney function (Parikh and Devarajan, 2008; Devarajan 2007). Although they have been used over decades and are applied for the evaluation of kidney function in the majority of studies, their application is limited: they lack sensitivity and specificity, especially in acute kidney injury and are influenced by multiple parameters such as muscle mass, liver function, and pharmacological substances (Parikh and Devarajan, 2008; Devarajan 2007). Thus, new biomarkers have been evaluated, such as cystatin c, human neutrophil gelatinase-associated lipocalin (NGAL), interleukin-18 (IL-18) and kidney-injury molecule 1 (KIM-1) (Parikh and Devarajan, 2008; Devarajan 2007; Belcher et al., 2011; Cruz and Goh, 2007). But so far only cystatin c measurements have in part been established for routine diagnostics.

The object of the present invention is to provide a reliable method which especially can be used in routine diagnostics and/or the monitoring of the renal function.

The inventors have surprisingly found out that special fragments of the proteoglycan Agrin, namely the C-terminal fragments which are derived by the cleavage of Agrin by the enzyme neurotrypsin can be used as reliable biomarker in the relevant diagnostic and screening applications in which a determination of the renal function is required.

Agrin is a large heparan proteoglycan with a molecular weight of 400-600 kDa. (Database accession number NP_{—}940978). The protein core consists of about 2000 amino acids and its mass is about 225 kDa. It is a multidomain protein composed of 9 K (Kunitz-type) domains, 2 LE (laminin-EGF-like) domains, one SEA (sperm protein, enterokinase and agrin) domain, 4 EG (epidermal growth factor-like) domains and 3 LG (laminin globular) domains (Bezakova and Ruegg, 2003).

Agrin exists in several splice variants and can be expressed as a secreted protein, containing the N-terminal NtA (N-terminal agrin) domain, which is the most abundant form of agrin and the predominant form expressed in motor neurons. It is produced in the soma of the neurons, transported down the axon and released from the axon ending of the motor nerve into the synaptic cleft of the NMJ (Bezakova and Ruegg, 2003). Here it acts as an agonist of LRP4 (low-density lipoprotein receptor-related protein 4) and may also become a component of the basal lamina (Kim et al., 2008). In the CNS (central nervous system), most agrin is expressed as a type-II transmembrane protein by alternative splicing at the N-terminus lacking the N-terminal NtA domain (Bezakova and Ruegg, 2003). Among neuronal and other tissues, agrin is highly expressed in the kidney, where it substantially contributes to the formation of the glomerular basement membrane (GBM) (Groffen et al., 1998a; Groffen et al. 1998b, Miner et al., 2011) possibly linking it to podocytes. These cells are part of the glomerulus, which is the organelle filtering small molecules and small proteins from the blood. The glomerulus consists of capillaries with fenestrated endothelium and the mesangial cells. These cells are modified smooth muscle cells that lie between the capillaries and the glomerulus. The purpose of these cells is to regulate blood flow and to secrete extracellular matrix to build up the glomerular basement membrane, prostaglandins, and cytokines. Podocytes are surrounding the capillaries by creating a large cellular surface due to lots of foot processes (pedicels). These pedicels express nephrins, which are membrane bound proteins capable as a filtration barrier between the blood stream and the primary urine in the Bowman's capsule (George, 2012). The characteristic of this filtration barrier is that large proteins or negatively charged proteins cannot permeate and remain in the blood stream. Low molecular weight proteins or metabolites are able to permeate and are transferred into the primary urine. A part of these substances is recycled in the nephron or degraded by cells of the tubular system or remains in the urine (Hausmann et al., 2012; Vallon, 2011).

Agrin is cleaved by an enzyme called neurotrypsin which plays an important role in controlling the activity of agrin. At present, agrin is the only known target of neurotrypsin. Neurotrypsin (Stephan et al 2008) cleaves agrin at 2 distinct sites called alpha- and beta-site. The alpha-site is located N-terminal from the SEA domain and the beta-site is placed in front of the LG3 domain of agrin. Cleavage at the alpha-site generates a ˜110 kDa C-terminal agrin fragment running at ˜130 kDa in a 4-12% bis-tris SDS gel. Cleavage at the beta-site liberates the C-terminal LG3 domain running at ˜22 kDa in the gel (Reif et al. 2007), also called CAF. In the C-terminal part of human agrin, there are 2 alternative splice sites y and z. At the y-site, there may be inserts of 0, 4, 17 or 21 (4+17) amino acids and at the z site there may be inserts of 0, 8, 11 or 19 (8+11) amino acids. Cleavage of agrin by neurotrypsin can generate in principle two different CAF containing agrin fragments. Besides full length agrin CAF and agrin C110 are agrin fragments that can be detected in blood. Additionally one can expect, that also the remaining fragment (app. kDa 90) without CAF obtained after cleavage at the beta-site of agrin C110 is present in blood.

The fragments, also depending on the presence of specific inserts at the y and z position, can have different functions especially in neuronal or muscular tissues (Bezakova and Ruegg, 2003). A list with the CAF containing agrin fragments derived by neurotrypsin cleavage is given in the table 1 below.

| Abbreviation | Description |
|---|---|
| C22, CAF | Naturally occurring 22 kd C-terminal agrin fragment generated by |
| | Neurotrypsin cleavage of Agrin at the beta cleavage site. The 22-kD |
| | corresponds to the apparent running position on PAGE gel. Insert at |
| | the Z position is not fixed. There can be no insert (CAF-z0) or |
| | inserts of 8 (CAF-z8), 11 (CAF-z11), or 19 (CAF-z19) amino acids, |
| | respectively. Species is not determined and could be from e.g. |
| | human-derived (human CAF), rat derived (rat CAF), mouse-derived |
| | (mouse CAF), chicken etc. For instance, human CAF-z8 represents |
| | the 22 kd C-terminal human-derived agrin fragment generated by |
| | Neurotrypsin cleavage of Agrin with an 8 amino acid insert at the Z |
| | position. |
| Agrin C110 | Naturally occurring C-terminal 110 kd agrin fragment generated by |
| | Neurotrypsin cleavage of Agrin at the alpha site. The 110-kD size |
| | corresponds to the apparent running position on PAGE gel. Can |
| | have inserts at the y position. Species is not determined and could |
| | be from e.g. human, rat, mouse, chicken etc. For instance, human |
| | C110-y4z8 represents the human derived C-terminal 110 kd agrin |
| | fragment having the 4 amino acid insert at the Y position and 8 |
| | amino insert at the Z position. |
| Ag | Full length agrin that is not cleaved at the alpha site or beta sites. |
| | Can have inserts at the y and z position. Species is not determined |
| | and could be from e.g. human, rat, mouse, chicken etc. For |
| | instance, human C110-y4z8 represents the human derived C- |
| | terminal 110 kd agrin fragment having the 4 amino acid insert at the |
| | Y position and 8 amino insert at the Z position. |

Circulating CAF and the agrin C110 fragment are detectable in human blood (Hettwer et al., 2013). The term CAF total if used in the following relates to the combined measurement of CAF (22 kDa) and agrin C110 and their added values.

Changes in kidney function may be associated with changes in CAF serum levels in human as CAF is small enough to penetrate the filtration barrier of the glomerulus. So far, OAF has never been explored as a novel marker of renal function.

Conventional serum renal function parameters are of limited use in the situation of acute kidney failure requiring hemodialysis or hemofiltration treatment, since they are removed by the procedure. Therefore they cannot be used to monitor the improvement or deterioration of kidney function in this setting. As the applicants point out in this application, CAF is so far the first renal biomarker reporting kidney function which is not influenced by hemodialysis.

As the following results and Figures show, especially CAF but also agrin C110 are promising new biomarkers for renal function. The levels of the new markers are highly correlated to kidney function (glomerular filtration rate), highly correlated with chronic kidney disease stages. CAF values or total CAF values above the normal range are indicative for a renal dysfunction. CAF may serve as a new tool for the early detection of DGF after kidney transplantation. A further important aspect is that CAF (22 kDa) levels are stable during hemodialysis and thus CAF is a valuable marker for kidney function of patients undergoing continuous dialysis or after transplantation of organs.

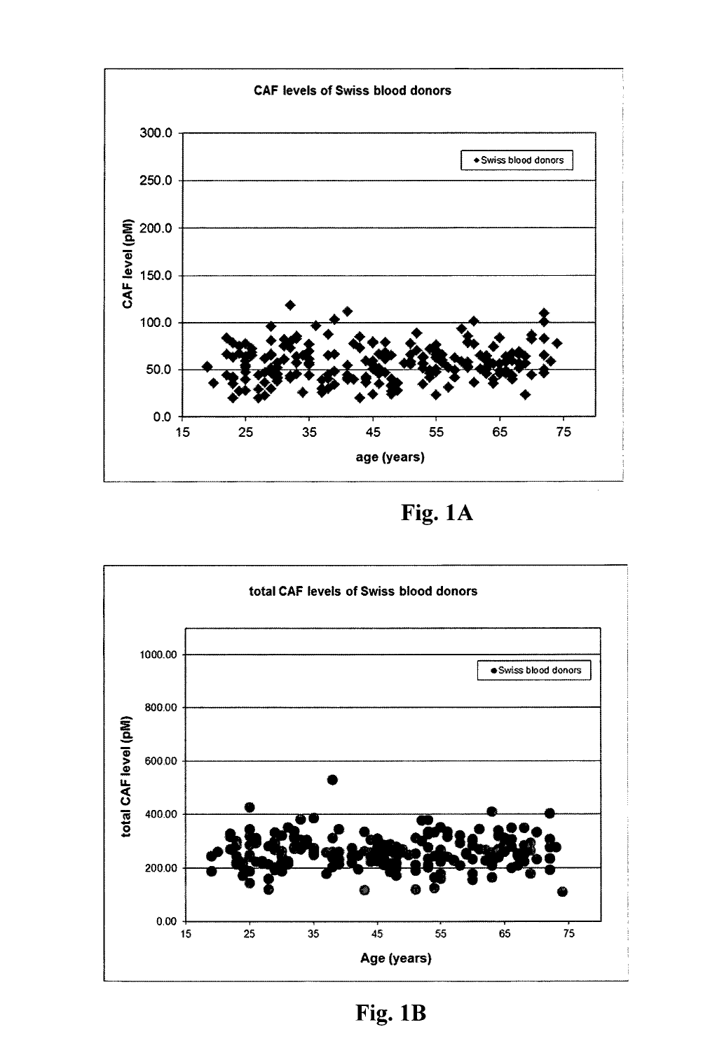

FIGS. 1A and B show the CAF and total CAF values of ostensibly healthy Swiss blood donors (n=210).

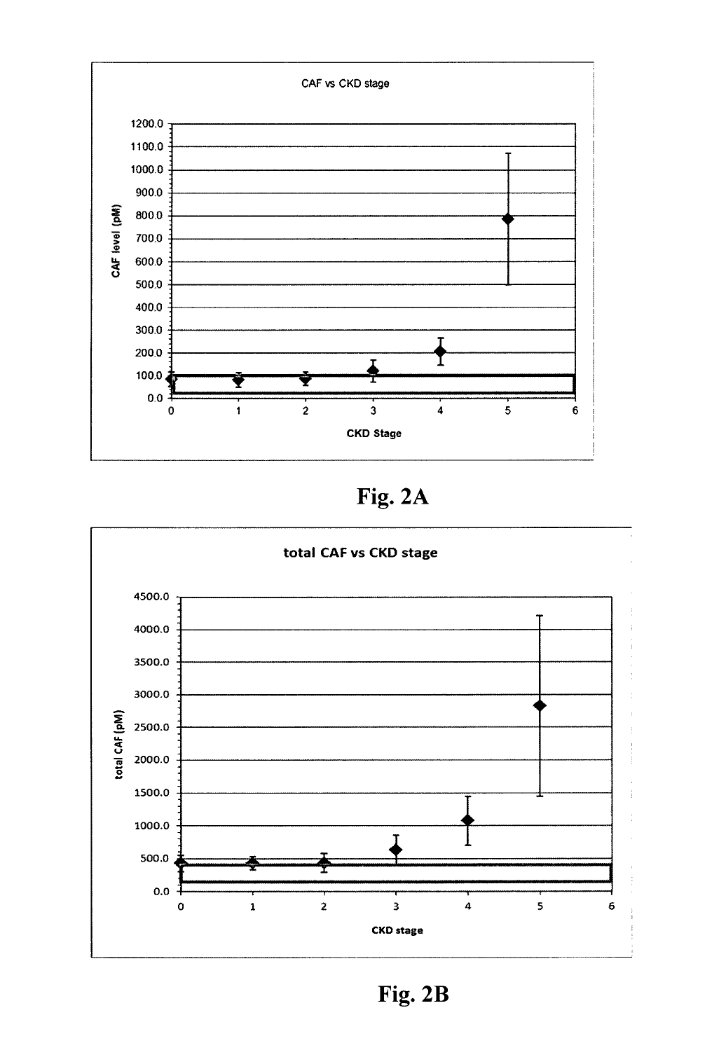

FIGS. 2A to C show the CAF, total CAF and NGAL values in dependence of chronic kidney disease (CKD) stages 0-5 (normal range for CAF and total CAF indicated by frame)

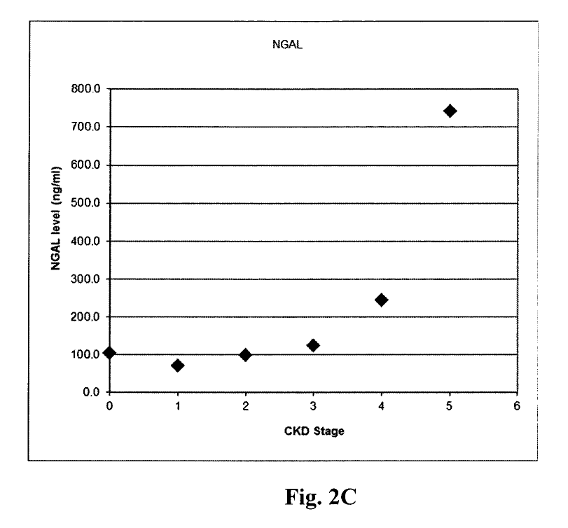

FIG. 3 shows the CAF levels before and after dialysis

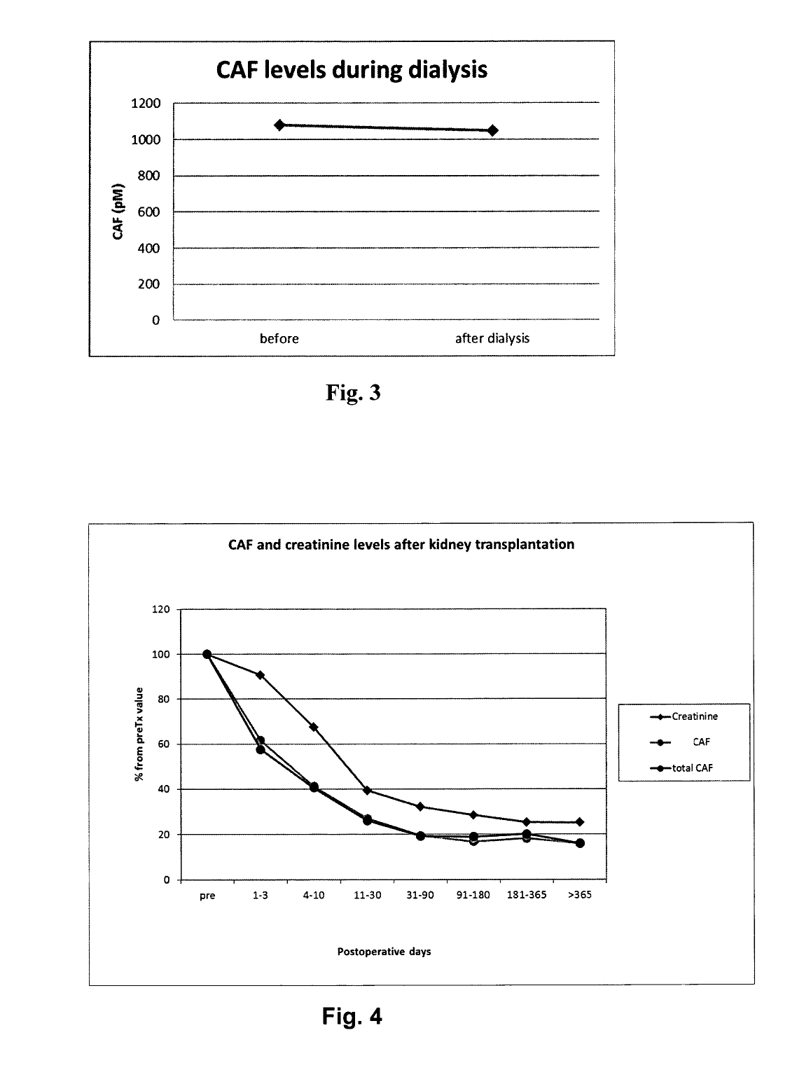

FIG. 4 shows the time course of CAF, total CAF and creatinine levels after kidney transplantation

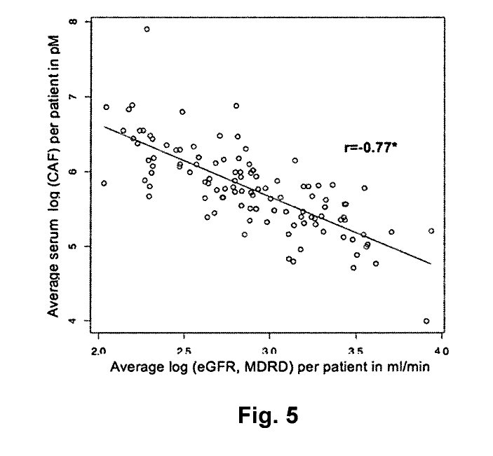

FIG. 5 shows the CAF and glomerular filtration rate (eGFR, MDRD)

### EXAMPLE 1

### Sandwich Elisa for CAF and totalCAF determinations

i) Microtiterplates

Microtiterplates (Maxi Sorp) coated with 125 μl per well with a solution of 0.5 μg/ml of antibodies (internal designation) 264E12B8, 264B12A8 as disclosed in EP 11000898 or 7H6, 4A7 or 8G7 as disclosed in a co-pending PCT-application claiming the same priority in Coating buffer (12 mM Potassium-phosphate Buffer, pH 7,4-pH 8,2; 137 mM NaCl; 2.7 mM KC) are used.

ii) Sample Preparation

For the assay undiluted serum samples are thawed at 37° C. for 10 minutes. The samples are mixed and centrifuged for two minutes at 14000×g. In order to prepare samples for 1 ELISA plate, 50 μl Sample incubation buffer (50 mM potassium-phosphate buffer, pH 7,2-8,2; 137 mM NaCl; 2.7 mM KC; 5 mg/ml HSA; 0-20% Glycerol; 0-50% (NH4)2SO4 and or 10-30% PEG 6000; 0.2-1% Tween 20) are added to the wells of a protein LoBind deep well plate. For the measurement of total CAF no PEG 6000 was added in the sample preparation buffer. Subsequently 50 μl serum sample are added to the same well and mixes with the incubation buffer by pipetting up and down 5-7 times. Additionally 50 μl of calibrator solution are added to some of the wells of the plate and treated in the same way as the sample. The preparation of calibrator is explained below.

The LoBind plate is tightly sealed with an adhesive cover foil and incubated exactly 30 min at 56° C. in a waterbath. The plate will float on the water surface, shaking is not desirable at this stage. After incubation a precipitate is generated and in order to pellet the precipitate, the plate is centrifuged for 5 min at 3000×g at room temperature. The cleared supernatant is diluted tenfold with Sample dilution buffer (50 mM Potassium-phosphate buffer pH 7,2-8,2; 137 mM NaCl; 2.7 mM KCl) by pipeting 90 μl Sample dilution buffer in the wells of the pre coated ELISA plate (MTP). 10 μl of the cleared supernatant from the LoBind deepwell plate are carefully transferred (without disturbing the pellet) into the precoated ELISA plate. The samples are mixed by pipeting up and down five to seven times. Then the MTP is tightly covered with an adhesive cover foil and incubated at room temperature (15-25° C.) for 1-16 h (or overnight).

iii) Calibrator Preparation

A thousand fold dilution of the CAF protein calibrator solution recombinant human Agrin C110 (without inserts ant the y and z sites) solution is prepared by making tenfold serial dilutions. The solution is diluted with Sample dilution buffer (50 mM Potassium-phosphate buffer pH 7,2-8,2; 137 mM NaCl; 2.7 mM KC) to a final concentration of 400 pM. This stock solution serves as source in the preparation of the 8 calibrator series.

To prepare the calibrator series for the assay, the volumes of the 400 pM CAF stock solution and Sample dilution buffer (as indicated in table 2) are pipetted into 1.5 ml protein LoBind tubes. Mix by flicking the tubes several times. As stated above 50 μl of the calibration series are added to the wells of the MTP and are then treated like the samples.

TABLE 2
Calibrator dilution series

| | | |
|---|---|---|
| calibrator | 400 pM CAF | Sample |
| concentration | protein calibrator | dilution buffer |

| | | | | | |
|---|---|---|---|---|---|
| 0 | pM | 0 | μl | 200 | μl |
| 20 | pM | 10 | μl | 190 | μl |
| 80 | pM | 40 | μl | 160 | μl |
| 140 | pM | 70 | μl | 130 | μl |
| 200 | pM | 100 | μl | 100 | μl |
| 260 | pM | 130 | μl | 70 | μl |
| 320 | pM | 160 | μl | 40 | μl |
| 380 | pM | 190 | μl | 10 | μl |

iv) ELISA Protocol

All further incubation steps on the plate (MTP) are carried out at room temperature 15-25° C. in the dark. Following the incubation (see ii) the wells of the MTP are washed 3 times with 300 μl Washing buffer (12 mM Potassium-phosphat Puffer pH 7,2-8,2; 137 mM NaCl; 2.7 mM KC; 0-2% Casein; 0-0.5% Tween 20). Between each washing step the MTP is incubated 1 min at room temperature. The washing fluid is then removed by aspirating or tapping. After the wash steps are completed, 100 μl CAF detector antibody solution, 28A6H11-biotin or 28H7G3-Biotin or 14B7B8-biotin Conjugate (0.01-2 mg/ml) are pipetted in all wells. The MTP is then incubated at room temperature for 30 min.

Subsequently the MTP is washed 3 times with washing buffer as described above and the washing fluid is removed by aspirating or tapping. Then 100 μl Streptavidin-polyHRP solution is pipetted in all wells and the MTP is incubated at room temperature for 30 min in the dark. The MTP is again washed 3 times with washing buffer as described above and the washing fluid is removed by aspirating or tapping. Then 100 μl TMB solution are pipetted in all wells and incubated at room temperature for 30 min in the dark. Finally, 100 μl Stop solution (Stop Reagent for TMB Microwell Substrates from SurModics) are pipetted in all wells and the optical density of each well at 450 nm is measured on a microplate reader. The yellow color is stable for 1 hour at room temperature. The above procedures are also part of the instructions of the NTCAF ELISA kit which can be purchased at www.neurotune.com

v) Results:

Evaluation of the Normal Range for CAF and Total CAF

To define the normal distribution of CAF, the ELISA assay was performed according to the above described procedure. 210 sera from ostensibly healthy Swiss blood donors, age 19-74 years, were analysed for the presence of CAF. The mean value was 56.8 pM±18.9 pM (mean value±standard deviation). This corresponds to a normal range of 18.9 pM up to 94.7 pM (2 standard deviations below the mean value up to 2 standard deviations above the mean value). The lowest value measured was 20 pM while the highest value was 118.4 pM. 8 individuals (3.8%) had CAF values above the normal range. The measurement of totalCAF (i.e. CAF plus the Agrin C110 fragment) of the same individuals resulted in mean CAF levels of 260 pM±59.6 pM and a normal range of 140.8 pM up to 379.2 pM. 3.3% of the people showed total CAF levels above the normal range. The results are shown in FIGS. 1A and B

CAF Levels in Patients Suffering from Diabetes Mellitus

189 patients suffering from diabetes mellitus II were analysed for CAF levels in serum. The mean CAF value observed was 81.4±29.5 pM. 24.3% of these patients had CAF levels above the normal range of 18.9 pM to 94.7 pM as defined from the healthy Swiss blood donors. It can be assumed that these patients have beginning kidney dysfunction.

CAF Levels in Dependence of the Chronic Kidney Disease Stage (CKD Stage)

205 patients in different stages of chronic kidney disease (CKD stages 1-5) were analysed for CAF and total CAF levels in blood. It became obvious that the CAF levels increase with the severeness of the kidney disease. This is in concordance to the NGAL measurements which were also performed with these patients. NGAL measurements were performed in a clinical diagnostic laboratory with state of the art analyzing methodology. Table 3 FIG. 2A to C summarize the results for CAF, total CAF and NGAL measurements.

TABLE 3
CAF, total CAF and NGAL in dependence of the CKD-stage

| CKD- | CAF | Total CAF | NGAL |
|---|---|---|---|
| stage | (pM) | (pM) | (ng/ml) |
| 0 | 83.3 | 428.8 | 104.6 |
| 1 | 81.5 | 431.5 | 70.5 |
| 2 | 87.0 | 434.0 | 98.2 |
| 3 | 120.0 | 627.6 | 125.1 |
| 4 | 205.7 | 1074.4 | 244.5 |
| 5 | 784.5 | 2824.1 | 720.8 |

Stability of the CAF Level During Hemodialysis

Due to its size (22 kDa) it is conceivable that low-flux and even high-flux dialyzers are unable to remove CAF from the patient's serum effectively. This is more obvious for the agrin C110 fragment. Furthermore, low serum CAF concentrations suggest that diffusion is unable to remove larger amounts of CAF or agrin C110 from the serum.

Serum from 14 patients undergoing hemodialysis with a Fresenius Fx60 membrane was analysed. One serum sample was taken immediately before dialysis and one sample was taken 5 minutes before the end of the dialysis. As the dialysis leads to a volume reduction of the patient's body fluid, CAF levels were corrected for this volume defect. As one can see from FIG. 3 the mean CAF value before dialysis was 1077 pM while the volume corrected CAF level after dialysis was 1047 pM. This is virtually identical. No loss of CAF during dialysis was observed. This is in contrast to the commonly used renal markers, e.g. creatinine and cystatin C (Huang et al., 2011).

CAF Level Reduction after Kidney Transplantation

Serum CAF levels of 110 end-stage renal disease (ESRD) patients undergoing kidney transplantation were analysed before and after transplantation. Serum creatinine levels were quantified using a well-established photometric measurement (Jaffe method, normal range 0.7-1.3 mg/dl in males and 0.5-1.1 mg/dl in females). Measurements were conducted at the institute of clinical chemistry at Klinikum rechts der Isar, Technische Universitat, Munich, Germany, central laboratory service. The serum creatinine levels served to calculate the glomerular filtration rate (eGFR). The correlation between serum CAF and creatinine levels/eGFR was calculated as within-patient-(cWP) and between-patient correlations (cBP). Moreover the association of CAF with delayed graft function (DGF) was assessed. The diagnostic value of CAF for early detection of DGF compared to creatinine was evaluated by receiver operating characteristics (ROC) analysis.

As one can see from FIG. 4A serum CAF levels strongly correlated with serum creatinine (r=0.86 (cWP), r=0.74 (cBP)) and eGFR (MDRD) (r=0.86 (cWP), r=0.77 (cBP)). Median pretransplant (pre-Tx) CAF levels were 19-fold higher than in healthy individuals (1115.0 pM vs. 56.6 pM). After transplantation, CAF levels decreased significantly faster than creatinine levels (postoperative day 1-3 (POD 1-3): 562.8 pM, i.e. 54% of pre-Tx levels, creatinine: pre-Tx 6.9 mg/dL, POD 1-3: 6.4 mg/dL, i.e. 93% of pre-Tx levels, p<0.001). Stable serum levels were reached 1-3 months after transplantation for CAF and creatinine (CAF: 145.1 pM, i.e. 13% of pre-Tx levels; creatinine: 1.6 mg/dL, i.e. 24% of pre-Tx levels). These levels are still elevated (about 1.5 times above normal level). This is in concordance with the fact that patients with a graft kidney do still suffer from a kidney dysfunction as they have only one functional kidney and not 2 fully functional kidneys as healthy people. OAF-levels at POD 1-3were significantly associated with DGF and outperformed creatinine in early detection of DGF (area under the curve (AUC)-CAF: 80.7% (95%-confidence interval: 72.3%-89.1%) vs. AUC-creatinine: 71.3% (95%-confidence interval: 61.8%-81.1%, p=0.061)).

In table 3 CAF and total CAF values for different patient groups are depicted.

TABLE 3
Mean CAF and mean total CAF values for different patient
groups sufferiung from kidney dysfunction.

| Patient | CAF | Total CAF |
|---|---|---|
| group | (pM) | (pM) |
| Pre- | 1115 | 4506 |
| transplantation | | |
| Dialysis | 1077 | 4237 |
| Diabetes | 81 | 293.8 |
| mellitus II | | |
| healthy | 56.8 | 260.0 |

Patients of the pre-transplantation group and the dialysis group suffer from CKD stage 5, which means total loss of kidney function. To survive, they have to be hemo-dialysed every 2-3 days. The increase in CAF and total CAF level is not reduced by this routine dialysis process. In the diabetes mellitus group, an elevation of the CAF and total CAF values could be observed. About 25% of these patients have CAF or total CAF values above the normal range. Naturally, this patient group is quite heterogeneous. As indicated above, CAF and total CAF increase with the progression of the disease (CKD stage).

Finally reference is made to FIG. 5 which shows that CAF and glomerular filtration rate (eGFR, MDRD) highly correlate. Logarithmic serum CAF levels were plotted against the logarithm of the estimated glomerular filtration rate (eGFR) calculated from serum creatinine levels using the “Modification of Diet in Renal Disease” formular (MDRD).

It can be seen that CAF correlates very well with the glomerular filtration rate (r=−0.7) quite similar as cystatin C does (r=−0.74, Tan et al., 2002) and thus, CAF can be confidentially considered to be a marker for kidney function.

### LITERATURE (IN ORDER OF APPEARANCE IN THE TEXT)

1. Parikh C R, Devarajan P. New biomarkers for acute kidney injury. Crit Care Med 2008; 36: 159-S165.
2. Devarajan P. Emerging biomarkers of acute kidney injury. Contrib Nephrol 2007; 156: 202-212.
3. Belcher K M, Edelstein C L, Parikh C R. Clinical applications of biomarkers for acute kidney injury. Am J Kid Dis 2011; 57: 930-940.
4. Cruz D N, Goh C Y. Early biomarkers of renal injury. Congest Heart Fail 2010; 16: 25-31
5. Bezakova G, Ruegg M A. New insights into the role of agrin. Nat Rev Mol Cell Biol 2003; 4: 295-308.
6. Kim N, Stiegler A L, Cameron T O, Hallock P T, Gomez A M, Huang J H, Hubbard S R, Dustin M L, Burden S J. Lrp4 is a receptor for Agrin and forms a complex with MuSK. Cell. 2008; 135(2): 334-42.
7. Groffen A, Ruegg M A, Dijkman H, van de Velden T J, Buskens C A, van den Born J, Assmann K J, Monnens L A, Veerkamp J H, van den Heuvel L P. Agrin Is a Major Heparan Sulfate Proteoglycan in the Human Glomerular Basement Membrane. J histochem&cytochem 1998; 46 (1): 19-27.
8. Groffen A J, Buskens C A, van Kuppevelt T H, Veerkamp J H, Monnens L A, van den Heuvel L P. Primary structure and high expression of human agrin in basement membranes of adult lund and kidney. Eur J Biochem 1998; 254: 123-128.
9. Miner J H. Glomerular basement membrane composition and the filtration barrier. Pediatr Nephrol 2011; 26: 1413-1417.
10. George B, Holzman L B. Signaling from the podocyte intercellular junction to the actin cytoskeleton. Semin Nephrol. 2012; 32(4): 307-18
11. Hausmann R, Grepl M, Knecht V, Moeller M J. The glomerular filtration barrier function: new concepts. Curr Opin Nephrol Hypertens. 2012; 21(4): 441-449.
12. Vallon V. The proximal tubule in the pathophysiology of the diabetic kidney. Am J Physiol Regul Integr Comp Physiol. 2011; 300(5): R1009-1022.
13. Stephan A, Mateos J M, Kozlov S V, Cinelli P, Kistler A D, Hettwer S, Rülicke T, Streit P, Kunz B, Sonderegger P. Neurotrypsin cleaves agrin locally at the synapse. FASEB J. 2008; 22(6): 1861-73.
14. Reif R, Sales S, Hettwer S, Dreier B, Gisler C, Wölfel J, Lüscher D, Zurlinden A, Stephan A, Ahmed S, Baici A, Ledermann B, Kunz B, Sonderegger P. Specific cleavage of agrin by neurotrypsin, a synaptic protease linked to mental retardation. FASEB J. 2007; 21(13): 3468-78.
15. Hettwer S, Dahinden P, Kucsera S, Farina C, Ahmed S, Fariello R, Drey M, Sieber C C, Vrijbloed J W. Elevated levels of a C-terminal agrin fragment identifies a new subset of sarcopenia patients. Exp Gerontol. 2013; 48(1): 69-75.
16. Huang S H, Filler G, Yasin A, Lindsay R M. Cystatin C reduction ratio depends on normalized blood liters processed and fluid removal during hemodialysis. Clin J Am Soc Nephrol. 2011; 6(2): 319-325.
17. Tan G D, Lewis A V, James T J, Altmann P, Taylor R P, Levy J C. Clinical usefulness of cystatin C for the estimation of glomerular filtration rate in type 1 diabetes: reproducibility and accuracy compared with standard measures and iohexol clearance. Diabetes Care 2002; 25(11):2004-9

1. A method for determining renal function for a patient, the method comprising:
taking a sample from the patient;
measuring an amount of at least one agrin fragment derived by neurotrypsin cleavage of agrin in the sample taken from a patient; and
determining renal function for the patient based upon the measured amount of the at least one agrin fragment in the sample.

2. The method according to claim 1, wherein the agrin fragment measured is CAF, agrin C110 or total CAF.

3. The method according to claim 1, wherein the sample is selected from the group consisting of blood, urine and spinal fluid.

4. The method according to claim 1 further comprising:
diagnosing whether the patient is afflicted with one or more renal diseases based upon the renal function determination.

5. The method according to claim 1 further comprising:
monitoring the renal function by repeating the taking, measuring and determining steps for the patient over a period of time.

6. (canceled)

7. The method according to claim 5, wherein monitoring is conducted after kidney transplantation, during hemodialysis, when the patient has been diagnosed with chronic kidney disease or the patient has suffered an acute kidney injury.

8. The method according to claim 1, wherein the determining step includes comparing the measured amount of the at least one agrin fragment in the sample taken from the patient to a reference sample comprising a known amount of an agrin fragment prepared by recombinant techniques.
